(19) United States
(12) Patent Application Publication
(10) Pub. No.: US 2023/0019881 A1
(43) Pub. Date: Jan. 19, 2023

(54) HONOKIOL BASED COMPOSITIONS AND METHODS FOR REDUCING CORTISOL LEVELS

(71) Applicant: Financial Summit Ventures, Inc., Las Vegas, NV (US)

(72) Inventors: Steven Adler, Cape Elizabeth, ME (US); Patrick T. Prendergast, Dublin (IE)

(73) Assignee: Financial Summit Ventures, Inc., Las Vegas, NV (US)

(21) Appl. No.: 17/955,119

(22) Filed: Sep. 28, 2022

Related U.S. Application Data
(63) Continuation-in-part of application No. 16/906,822, filed on Jun. 19, 2020, now abandoned.
(60) Provisional application No. 62/985,417, filed on Mar. 5, 2020.
(60) Provisional application No. 62/864,171, filed on Jun. 20, 2019.

(51) Int. Cl. A61K 31/05; A61K 36/63; A61K 36/81; A61K 31/685; A61K 45/06; A61K 36/42; A61K 36/575

(52) U.S. Cl. CPC A61K 31/05; A61K 31/685; A61K 36/42; A61K 36/63; A61K 36/81; A61K 36/575; A61K 45/06

(57) ABSTRACT

Compositions including plant-derived components, optionally administered with CBD, an amount of one or both of magnolol and honokiol effective to provide an antiangiogenic or anti-inflammatory effect that may be useful for reducing cortisol levels and treating a variety of ailments. Furthermore, methods of antiangiogenic or anti-inflammatory and reducing cortisol levels in a human. Plant-derived components of such compositions include magnolol and honokiol as extracts or compounds, or tinctures or solutions and may also include Ashwagandha and/or Magnolia Bark. The compositions may also include other therapeutic compounds such hydroxytyrosol (such as that in olive oil), phospholipids, and carotenoids. The present invention includes methods of treatment of various ailments including anxiety, depression, post-traumatic stress disorder (“PTSD”), stress, insomnia, and drug addiction. The methods may also be used to reduce body weight and/or enhance alertness and focus.

### CROSS REFERENCE TO RELATED APPLICATIONS

This application is a continuation-in-part of U.S. Application No. 16/906,822, filed Jun. 19, 2020 and titled PLANT-BASED COMPOSITIONS AND METHODS FOR REDUCING CORTISOL LEVELS, which claims priority to expired Provisional U.S. Application No. 62/985,417, filed Mar. 5, 2020, and expired Provisional U.S. Application No. 62/864,171, filed Jun. 20, 2019, the entire disclosures of each of which are incorporated herein by reference.

### FIELD OF THE DISCLOSURE

The present disclosure relates to methods and compositions for reducing cortisol levels in an animal. Specifically, the present invention relates to plant-based compositions, which may include one or both of magnolol and honokiol in combination with a cannabidiol, and methods for administering such compositions to reduce cortisol levels in an animal such as a human. Furthermore, the present disclosure relates to methods of using plant-based compositions, which may include cannabidiol, to treat various disorders

### BACKGROUND OF THE DISCLOSURE

Cannabidiol (CBD) is known to be used as a treatment for many common ailments. CBD is one of the chemical compounds, referred to as cannabinoids, found in the cannabis or marijuana plant, Cannabis sativa. Tetrahydrocannabinol (THC) is a psychoactive component of cannabis, but CBD is not psychoactive, which makes it an appealing option for providing relief from pain and other symptoms without the mind-altering effects of marijuana or pharmaceutical drugs.

Generally, CBD oil is made by extracting CBD from the cannabis plant, then diluting the CBD with a carrier oil such as, for example, coconut oil or hemp seed oil. Although CBD oil has been used to relieve chronic pain and offset anxiety, the physiological effects on the body may be different depending upon how the CBD is introduced into the body.

In general, sustained high levels of cortisol may interfere with endocrine and immune system function. High cortisol levels have also been linked to disorders in the body, including the destruction of healthy muscle and bone and reduced rates of healing and cell regeneration. As such, compositions that reduce cortisol levels in animals, and in particular, humans, may be useful.

### SUMMARY OF THE DISCLOSURE

Accordingly, the present invention provides compositions including plant-derived components, optionally administered with CBD, which may be useful for reducing cortisol levels and treating a variety of ailments. Furthermore, methods of reducing cortisol levels in a human and methods of treating various ailments using a composition described herein are also provided. Plant-derived components of such compositions may include extracts or compounds from Ashwagandha and/or Magnolia Bark. The compositions may also include other therapeutic compounds such hydroxytyrosol (such as that in olive oil), phospholipids, and carotenoids.

The present invention includes methods for reducing a level of cortisol in a subject, including waking cortisol levels. Such methods may further include attenuating the Cortisol Awakening Response. The compositions may assist in maintaining a proper level of cortisol sufficient for conversion of proteins into energy for a limited duration and may release glycogen to thereby counteract inflammation in the human body and also offset sustained high levels of cortisol sufficient to negatively affect the human body.

The present invention further includes methods of treatment of various ailments including anxiety, depression, post-traumatic stress disorder (“PTSD”), stress, insomnia, and drug addiction. The methods may also be used to reduce body weight and/or enhance alertness and focus.

### BRIEF DESCRIPTION OF THE FIGURE

FIG. 1 illustrates a transdermal patch according to an embodiment of the invention.

FIG. 2 is a graph showing the mean difference in CAR levels over a study period compared to the baseline condition.

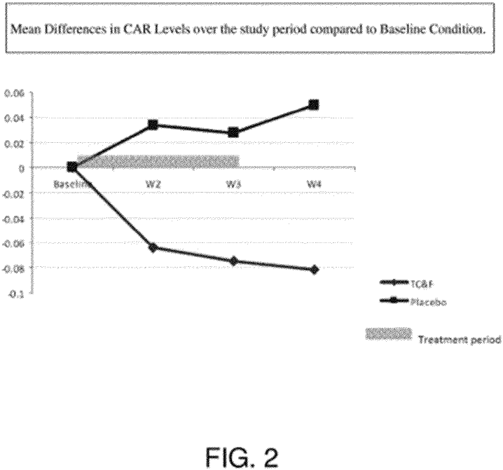

FIG. 3 is a graph showing the mean waking cortisol levels over a study period.

FIG. 4 is a graph showing the changes in daily diary questionnaire measures of psychological well-being over the course of a study.

### DETAILED DESCRIPTION OF EMBODIMENTS OF THE INVENTION

The present invention provides compositions including plant-derived components, optionally administered with CBD, an amount of one or both of magnolol and honokiol effective to provide an antiangiogenic or anti-inflammatory effect that may be useful for reducing cortisol levels and treating a variety of ailments. Furthermore, methods of antiangiogenic or anti-inflammatory and reducing cortisol levels in a human. Plant-derived components of such compositions include magnolol and honokiol as extracts or compounds, or tinctures or solutions and may also include Ashwagandha and/or Magnolia Bark. The compositions may also include other therapeutic compounds such hydroxytyrosol (such as that in olive oil), phospholipids, and carotenoids. The present invention includes methods of treatment of various ailments including anxiety, depression, post-traumatic stress disorder (“PTSD”), stress, insomnia, and drug addiction. The methods may also be used to reduce body weight and/or enhance alertness and focus.

The present invention is explained in greater detail below. This description is not intended to be a detailed catalog of all the different ways in which the invention may be implemented, or all the features that may be added to the instant invention. For example, features described with respect to one embodiment may be incorporated into other embodiments, and features described with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition includes components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

As used throughout this application, the word “may” is used in a permissive sense (i.e., meaning having the potential to), rather than the mandatory sense (i.e., meaning must). Similarly, the words “include”, “including”, and “includes” mean including but not limited to.

Furthermore, the term “about,” as used herein when referring to a measurable value such as an amount of a compound or agent of this invention, dose, time, temperature, and the like, is meant to encompass variations of ± 10%, ± 5%, ± 1%, ± 0.5%, or even ± 0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

“Pharmaceutically acceptable” refers to those properties and/or substances that are acceptable to the patient from a pharmacological and/or toxicological point of view, and/or to the manufacturing pharmaceutical chemist from a physical and/or chemical point of view regarding composition, formulation, stability, patient acceptance, bioavailability and compatibility with other ingredients.

“Pharmaceutically acceptable salt” refers to an acid or base salt of a compound of the invention, which salt possesses the desired pharmacological activity and is neither biologically nor otherwise undesirable. As used herein, when a compound is referenced, it is to be understood that the compound name includes any pharmaceutically acceptable salts thereof, regardless of whether the pharmaceutically acceptable salts are specifically referenced.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, fifteen-fold, etc.

The term “inhibit” or “reduce” or grammatical variations thereof as used herein refers to a decrease or diminishment in the specified level or activity, such as about 10%, 15%, 25%, 35%, 40%, 50%, 60%, 75%, 80%, 90%, 95% or more.

“Subject” refers to an animal such as a mammal, and in particular, a human. A mammal or human subject may also be referred to as an “individual.”

“Mammal” refers to a warm-blooded vertebrate animal with hair or fur. Examples include without limitation members of the human, equine, porcine, bovine, murine, canine or feline species.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease, condition, or ailment. Including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. For methods of reducing a level of cortisol, a therapeutically effective - amount may reduce the level of cortisol in the subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

By the term “treat,” “treating,” or “treatment of” (or grammatically equivalent terms), it is meant that the severity of the subject’s condition is reduced or at least partially improved or ameliorated and/or that some alleviation, mitigation or decrease in at least one clinical symptom is achieved. “Treating” in reference to a disease, disorder, ailment, or condition may refer to: (i) inhibiting a disease, disorder or condition, e.g., arresting its development; and/or (ii) relieving a disease, disorder, ailment, or condition, e.g., causing regression of the clinical symptoms.

The term “administering” or “administration” of a compound and/or composition of the present invention to a subject includes a route of introducing or delivering to a subject a compound to perform its intended function. Administration includes self-administration and the administration by another.

“Concurrently” means sufficiently close in time to produce a combined effect (that is, concurrently can be simultaneously, or it can be two or more events occurring within a short time period before or after each other). In some embodiments, the administration of two or more compounds or compositions “concurrently” means that the two compounds/compositions are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

“Krebs Cycle,” as used herein, means a series of enzymatic reactions in aerobic organisms involving oxidative metabolism of acetyl units and producing high-energy phosphate compounds such as ATP, which serve as the main source of cellular energy. A Krebs Cycle may be referred to, or include a citric acid cycle, or a tricarboxylic acid cycle.

### Compositions According to Embodiments of the Invention

Provided according to embodiments of the invention are plant-based compositions that include: (1) olive oil(s) and/or a phytochemical therein; (2) one or more phospholipids; (3) one or more carotenoids; (4) Ashwagandha and/or an active compound therein; (4) Magnolia Bark and/or an active compound therein; or (5) any combination thereof. In particular embodiments, the one or more phospholipids include lecithin and/or a glycerophospholipid such as phosphatidylserine. In other embodiments of the invention, the compositions include: (1) one or more carotenoid; (2) one or more phospholipids, (3) medium chain triglyceride (MCT) oil(s); (4) Magnolia Bark extract (and/or a compound therein); or (5) any combination thereof. Such compositions may be useful for decreasing a level of cortisol in a subject. Cortisol is a steroid hormone that is primarily produced in the adrenal gland.

“Olive Oil,” as used herein, refers to a fixed oil expressed from olive fruits. Olive oil is generally composed of esters of oleic acid, forming approximately 80% of the total fatty acid content. Linoleic acid and saturated palmitic acid generally form the balance of the fatty acid content, as well as one or more of: tocopherols, beta-carotene, phytosterols, pigments, terpenic acids, flavonoids (e.g., luteolin, quercetin, squalene), and phenolic compounds (e.g., oleuropein, tyrosol). Hydroxytyrosol, 2-(3,4-dihydroxyphenyl)-ethanol, is a phytochemical component of olive oil. In some particular embodiments, an olive oil includes hydroxytyrosol at a concentration in a range of about 375 to 700 umol/kg olive oil, and in particular embodiments, at a concentration in a range of about 400 to 500 umol/kg. In some embodiments of the invention, a phytochemical such as hydroxytyrosol may be included in the composition in lieu of or in addition to the olive oil.

“Phospholipids” are a type of phosphorous-containing lipid. In general, phospholipids are polar lipids that contain a glycerol molecule with a phosphate group attached through the ester bond to one carbon and two hydrocarbon tails. In some embodiments, the phospholipid includes a glycerophospholipid such as phosphatidylserine. In some embodiments, the phospholipid includes lecithin, which includes a mixture of phospholipids such as phosphatidylcholine, phosphatidylethanolamine, and phosphatidylinositol. In some embodiments, phospholipids such as phosphatidylserine may be useful to assist brain cells metabolizing glucose and to bind with neurotransmitters.

“Carotenoids” are yellow, orange, and red pigments produced by plants and algae. One example is astaxanthin (CAS 472-61-7). Another example is β-carotene (CAS 7235-40-7).

“Ashwagandha,” refers to Withania Somnifera, a plant in the nightshade family. The root powder or extracts may be used in compositions discussed herein and may act as an adaptogen. Compounds in Ashwagandha that are active include at least one withanolide. Withanolides are a group of chemical compounds naturally occurring in plants. Structurally, withanolides consist of a steroid backbone bound to a lactone or one of its derivatives. One particular withanolide that may be useful in embodiments herein is Withaferin A (CAS 5119-48-2). Withanolides such as Withaferin A may be useful to down regulate vascular endothelial growth factor (VEGF) gene expression in Ehrlich ascites tumor cells. Withaferin A may also be administered to inhibit COX-2 and promote anti-inflammatory activity. In the present invention, a withanolide may also be useful to interact with cortisol receptors as antagonists and neutralize the adverse effects of cortisol.

“Magnolia Bark Extract,” as used herein, means an herbal extract made from the bark of the Magnolia officinalis tree. The bark extract contains at least two active phytochemicals: honokiol and magnolol. Honokiol may provide an anti-stress effect and magnolol may provide an anti-depressant effect.

The compositions of the invention may include any suitable amount of each therapeutic agent described above. In general, any amount that provides a therapeutic effect described herein may be used. Particular compositions of the invention include 20 to 30 wt% (weight percent) of Ashwagandha extract or powder (having withanolides at more than 5%); 45 to 55 wt% of lecithin; 5 to 15 wt% phosphatidylserine; 5 to 15% olive oil containing hydroxytyrosol (e.g., 400-500 umol hydroxytyrosol/kg); and 1 to 3 wt% Magnolia bark or Magnolia Bark extract. Another particular composition of the invention includes 5 to 15 wt% phosphatidylserine; 5 to 15 wt% olive oil containing hydroxytyrosol; 0.02 to 0.4 wt% P-carotene; 70 to 80% lecithin; and 3 to 10% magnolia bark extract. Another composition according to an embodiment of the invention includes astaxanthin at 2 to 5 wt%, lecithin at 65 to 75 wt%; phosphatidylserine at 10 to 15 wt%; medium chain triglyceride oil (MCT oil) at 10 to 14 wt%; 0.1 to 0.5 wt% honokiol; and 0.1 to 0.5 wt% of one or both of magnolol and honokiol. In all embodiments described above, the weight percentages are relative to the total weight of the therapeutic ingredients and do not reflect carrier liquid or solvent.

Additionally, in some embodiments, one or both of magnolol and honokiol may be included as a small-molecule polyphenol isolated from a flowering plant in the sub-family magnolioideae of the genus Magnolia and other related polyphenols, such as, for example, magnolol. One or both of magnolol and honokiol is included in amounts effective to provide antiangiogenic, anti-inflammatory, and antitumor properties and/or anti-cancer effects. The present invention provides for anti-cancer treatments that include effective amounts of honokiol in combination with more conventional cancer treatments, such as, by way of non-limiting example chemotherapy and radiation treatments. Honokiol may also be included to treat systemic cancers that are difficult to treat with radiation.

According to the present invention one or both of magnolol and honokiol is administered in effective amounts (in combination with the other active agents described to block signaling in tumors. Some embodiments include blocking signaling in cells with defective p53 function and activated by directly blocking the activation of phospholipase D.

In another aspect of the present invention, honokiol may be administered in effective amounts to induce cyclophilin D, and potentiate mitochondrial permeability transition pore. Administered honokiol is sustained until if causes destruction of cells with a wild-type p53.

In some embodiments, one or both of magnolol and honokiol may be administered orally as a tincture or aqueous solution. Amounts of one or both of magnolol and honokiol may be administered that are sufficient to modify neuronal activities. Neuronal activities may include suppression of seizure activity induced by intraventricular injection of penicillin G and central muscle relaxation. One or both of magnolol and honokiol may also be administered in amounts effective to inhibit thrombosis by inhibiting thromboxane formation and intracellular calcium mobilization in platelets. Still further embodiments may include administration of tinctures or solutions in amounts effect to scavenge one or more of: hydroxyl radicals, lipid peroxidation, or reduce acetyl-CoA:1-alkyl-sn-glycero-3-phosphocholine acetyltransferase activity.

In some embodiments, one or both of one or both of magnolol and honokiol may be administered via a tincture or solution in an amount effective to induce apoptosis in SVR angiosarcoma cells and beneficially reduce phosphorylation of MAP kinase, akt, and c-src. The administered magnolol and/or honokiol may additionally modify antiangiogenic activity and block phosphorylation and rac activation due to VEGF-VEGFR2 interactions.

Still further implementations of the present invention include administration of effective amounts of one or both of magnolol and honokiol to cause apoptosis in CLL cells via activation of caspase 8, followed by caspase 9 and 3 activation and prevent interleukin-4-mediated survival of CLL cells, and potentiated the cytotoxicity of chlorambucil, fludarabine, and cladribine. Additionally, one or both of magnolol and honokiol may be administered via tincture and/or solution in amounts sufficient to adversely affect myeloma cells not adversely affect PBMCs. Administration of one or both of magnolol and honokiol be continued until it induces release of mitochondrial proapoptotic protein AIF to the cytosol. The magnolol and honokiol may be a precursor to apoptosis in the presence of IGF-1, interleukin-6, bone marrow stroma, and prevented phosphorylation of Akt, Stat-3, and Erk2 and act as an upstream target of action.

In some embodiments, one or both of one or both of magnolol and honokiol may be administered in an amount and frequency sufficient to induce expression of the mitochondrial protein cyclophilin D and blockade of cyclophilin D by siRNA or cyclosporin A and increase outer mitochondrial permeability and induce reactive oxygen.

Administration of one or both of one or both of magnolol and honokiol may be continued until the administration affects NF-_{K}B signaling and blocks NF-_{K}B and Akt activation as a result of TNF-a stimulation, enhancing TNF-a-mediated cell death and blocks production of TNF-a, MCP-1, interleukin-8, and ICAM-1.

In some embodiments of the invention, additional compounds may be added to the compositions for additional therapeutic effects. Such compounds include CMT oil, omega-3 fatty acids, such as docosahexaenoic acid, ethanol; antioxidants, and polyphenols such as a proanthocyanidin, including those derived from the grape or seed of Vitis vinifera. Additionally, in some embodiments, the compositions include at least one mineral such as calcium, copper, iron, iodine, lithium, magnesium, manganese, potassium, vanadium, and zinc. The minerals may act as Krebs Cycle intermediates. In particular embodiments, magnesium, calcium, zinc and iron may act as Krebs Cycle intermediates. In some embodiments, the compositions further include a B-complex vitamin such as Vitamin B1, Vitamin B2, Vitamin B3, Vitamin B5, and/or Vitamin B6. Further, in some embodiments, the compositions include the herb Luo Han Guo (Siraitia grosvenorii), which may be particularly useful for methods that include treatment of drug addiction.

In some embodiments of the invention, the therapeutic composition may be dissolved or dispersed in a carrier liquid or solvent. The concentration of the composition in the carrier liquid or solvent may vary depending on the mode of administration, type of ailment treated or method performed, and the particular patient being treated. However, in some embodiments, a concentration of the total therapeutic composition in a carrier liquid/solvent may be, for example, in a range of 0.375 mg/ml to 1.5 mg/ml up to about 3 mg/ml to 30 mg/ml. Any suitable carrier liquid or solvent may be used, and this may be greatly dependent on the mode of administration. However, in particular examples, the carrier liquid/solvent includes propylene glycol, vegetable glycerin (glycerine), or a combination of propylene glycol and vegetable glycerin. As used herein, a therapeutic composition dissolved or dispersed in a carrier liquid or solvent is also considered a composition of the invention.

Compositions according to embodiments of the invention do not necessarily include CBD. However, in some embodiments, the compositions described above may further include CBD or its derivatives, which includes the full spectrum of hemp oil products, including but not limited to, cannabidiol with little affinity for CB1 or CB2 receptors; CBD-C1 (Cannabidiol-C1); CBD-C4 (Cannabidiol-C4); CBDA (Cannabidiolic Acid); CBG (Cannabigerol) CBGA (Cannabigerolic Acid); CBC (Cannabichromene); CBL (Cannabicyclol); CBT (Cannabitriol); CBE (Cannabielsoin); CBR (Cannabiripsol); DCBF (Dehydrocannabifuran); CBF (Cannabifuran); CBCN (Cannabichromanon); CBT (Cannabicitran); CBCA (Cannabichromenic Acid); CBLA (Cannabicyclolic Acid); CBEA-A (Cannabielsoic Acid A); CBEA-B (Cannabielsoic Acid B); CBNA (Cannabinolic Acid); CBV (Cannabivarin); THCV (Tetrahydrocannabivarin); CBDV (Cannabidivarin); CBCV (Cannabichomevarin); CBGV (Cannabigerovarin); CBLV (Cannabicyclovarin); CBVD (Cannabinodivarin); and CBTV (Cannabitriolvarin). Although any suitable amount of CBD or its derivatives may be included in compositions and methods herein, in some embodiments, the amount of CBD per treatment may be within a range of about 5 mg to about 100 mg. Preferred embodiments may administer a dose between about 10 mg to about 20 mg from one to four times daily.

Any suitable pharmaceutical formulation or dosage form may be used to administer the compositions of the invention. Such forms include, without limitation, tablets, capsules, dragees, liquid preparations and solutions, troches, lozenges, melts, powders, micronized particles, non-micronized particles, solutions, emulsions, elixirs, suspensions, syrups or inhalations and controlled release forms thereof. In some embodiments, all of the therapeutic compounds or components are present in one dosage form, while in other embodiments, the therapeutic compounds or components are formulated and administered in separate dosage forms.

In particular embodiments, one or more of the therapeutic compounds or components are provided in a form suitable for oral administration, such as a capsule (e.g., a gelatin capsule). In other embodiments, one or more of the therapeutic compounds or components are provided in a form suitable for inhalation, e.g., via vaping. In some embodiments, one or more compositions of the invention are included within a thin film strip. In particular embodiments, a thin film strip may include a polyolefin film with an acrylic polymer adhesive. In some cases, the thin film is a monolithic patch design that does not include a reservoir. The proper dosage may be determined according to a size of a dosing matrix included in the patch. Further examples include transdermal ointments and solutions, a transdermal patch, a subcutaneous insertion device (with or without a pump), and/or a mucous membrane film.

### Methods of Administration and Treatment

Provided according to embodiments of the invention are methods of administering a composition of the invention to a subject in need thereof. A subject is ‘in need thereof’ if they are in need or desirous of the therapeutic effects or benefits of the inventive methods. In further embodiments, methods include administering a composition of the invention concurrently or sequentially with CBD or a derivative thereof. CBD and its derivatives include the full hemp oil spectrum described above.

Administration of compositions of the invention may be accomplished via various mechanisms based upon an amount of the composition to be administered, the length of time for the administration, the situs for administration, the capability of a patient receiving the therapeutic combination, the caregiver choice and/or the desired effect to be produced. In some embodiments, the administration regimen may include pretreatment and/or co-administration with at least one additional therapeutic agent, either a therapeutic agent described above or another therapeutic agent.

The compositions of embodiments of the invention can be administered to a subject in any of a wide range of routes. It is within the skill of the ordinary artisans to select a specific formulation and route of administration. Administration may be systemic or local. By way of example but not limitation, suitable routes include enteric, intravenous, parenteral, topical, transdermal, oral, rectal, ocular, nasal or vaginal routes. Parenteral routes include subcutaneous, intramuscular, intravenous, intraperitoneal, intradermal and sublingual administration. Also, compositions may be implanted into a patient or injected using a drug delivery system which can slowly release the drug over an extended time frame utilizing drug slow release formulations already available in the art.

In particular embodiments, the compositions are administered orally, for example via a capsule, tablet, liquid preparations and solutions, troches, lozenges, melts, powders, micronized particles, non-micronized particles, solutions, emulsions, elixirs, suspensions, and/or syrups.

In other particular embodiments, the compositions are administered in vapor form via an inhalable steam or mist, a mini vape, an Advanced Personal Vaporizer, a Vape Pen, and/or a covered vaporizer. Vaporizers may be selected according to an amount of vapor cloud to be produced. For example, a vaporizer of between about 1 ohm to 6 ohms may be used for little to moderate vapor cloud production, wherein the vapor cloud includes active agents from the therapeutic compositions. A vaporizer of less than 1 ohm may be used to generate larger amounts of vapor. In addition, those therapeutic compositions selected to be administered via a vapor producing apparatus may be further combined with a carrier liquid, such as propylene glycol, vegetable glycerin or a combination of propylene glycol and vegetable glycerin.

In yet other particular embodiments, the compositions of the invention are administered via a transdermal patch.

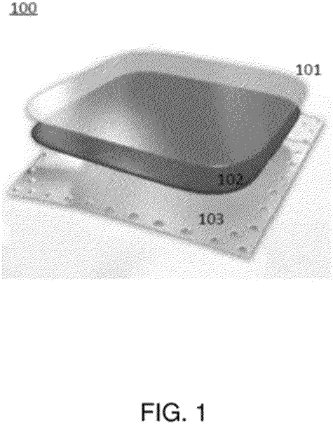

Referring now to FIG. 1, an exemplary transdermal patch 100 is illustrated. The transdermal patch 100 may include a clear (or opaque) polyolefin layer 101 that covers and protects a dosing matrix 102. The dosing matrix may be determined according to the dosage to be administered, the body weight of a patient, the length of time for the therapeutic agents to enter the bloodstream, and the area available to cover with the patch (for example, patch dimensions for application to a torso may be larger than a patch for application on a leg, which may in turn be larger than a patch for application to an arm). The dosage matrix may also vary but, in some embodiments, may be between about 0.5 inches by 0.5 inches to about 3.0 inches by 3.0 inches. Rectangular, oval, circular, or other arcuate shapes or polygons may also be deployed for one or both of the patch shape and the dosage matrix 102 shape.

In some embodiments of the invention, the dosage matrix may include between about 10 mg and 300 mg of a therapeutic composition including one or more of CBD, CBD oil, the plant-based compositions described herein, or a combination thereof. The therapeutic agents may effectively be directed to the bloodstream of the subject following application of the adhesive to a portion of the subject’s skin, thereby bypassing the digestive tract and/or mucous membranes.

In some cases, the transdermal patch 100 with an adhesive layer 103

5 to affix the patch to a patient’s skin in a manner suitable for transdermal administration of the active agents described herein and may include printed language and/or a symbol, a pictorial representation, a hash, a barcode or other device that is descriptive of the therapeutic combination included in the dosage matrix. Application instructions and other information may also be included. In particular, embodiments, a transdermal patch according to the present invention may be free of DMSO and only include GRAS approved adhesives and materials. Other embodiments may utilize traditional DMSO type patches.

Any suitable regimen for administering the compositions may be used, and the regimen may vary greatly depending on the mode of administration, the subject, and the reasons for treatment. For example, administration of the compositions may be performed once daily, twice daily, or any at any other suitable time frame. Additionally, as described above, when the regimen includes co-administration with CBD or its derivatives, the plant-based compositions according to the invention may include CBD or its derivatives; the plant-based compositions of the invention may be administered concurrently or sequentially with CBD or its derivatives; or the plant-based compositions of the invention may be administered on a separate schedule than CBD or its derivatives.

The plant-based compositions according to invention, with or without CBD or its derivatives, may provide for reduced cortisol levels in the subject. As such, in some embodiments of the invention, methods include administering to a subject a composition of the invention to reduce a level of cortisol in the subject. In some embodiments, methods include administering a composition of the invention to a subject to attenuate the subject’s Cortisol Awakening Response (CAR), which is the rapid rise in cortisol levels immediately following wakening. Further, in some embodiments, methods include administering a composition of the invention to a subject to reduce waking cortisol levels in the subject.

In some embodiments of the invention, methods include administering compositions of the invention to treat various other ailments, including anxiety, depression, post-traumatic stress disorder (“PTSD”), stress or psychological strain, insomnia, and drug addiction. Additional methods include administering compositions of the invention to decrease a body weight in a subject and/or to enhance brain function, such as alertness or focus, in the subject. The compositions may also be used to treat patients with long term and/or terminal illness, both to relieve one or more symptoms of the illness (e.g., nausea, fatigue, discomfort) and to relieve anxiety that may result from being ill. Furthermore, in some embodiments of the invention, methods include administering a composition of the invention to reduce inflammation due to arthritis, ameliorate symptoms and/or inhibit the progression of one or more of tumors, tuberculosis, asthma, bronchitis, orthopedic discomfort, fibromyalgia, menstrual functioning, hiccups, and liver disease.

Having described the present invention, the same will be explained in greater detail in the following examples, which are included herein for illustration purposes only, and which are not intended to be limiting to the invention.

### EXAMPLES

### Example 1: Plant Extract Formulations

Specific formulations according to embodiments of the invention are listed below. These formulations may be administered alone or in combination with CBD or derivatives thereof.
Formulation 1:
Ashwagandha (125 mg)
Lecithin (250 mg)
Phosphatidylserine (50 mg)
Olive Oil supplemented to between 400-500 umol hydroxytyrosol/kg oil (e.g., 50 mg)
Magnolia Bark Extract (Magnolol and Honokiol; 7 mg)
Formulation 2:
Astaxanthin (10 mg)
Lecithin (250 mg)
Phosphatidylserine (50 mg)
Medium Chain Triglyceride Oil (50 mg) , or alternatively olive oil
Honokiol (1 mg)
Magnolol (1 mg)
Formulation 3:
125 mg of Ashwagandha (Withanolides at or more than 5 %);
250 mg or more or lecithin,
50 mg or more of Phosphatidylserine;
50 mg or more of olive oil containing hydroxytyrosol; and
7 mg or more of magnolia bark or magnolia bark extract.
Formulation 4:
50 mg or more of Phosphatidylserine (~70% or more);
50 mg or more of olive oil containing hydroxytyrosol;
500 IU of Betacarotene;
350 mg of lecithin; and
25 mg or more of magnolia bark extract (~40% or more).

Additional components may include one or more of the following: 25 mg or more of Phosphatidylcholine; 25 mg or more of docosahexaenoic acid: 25 mg or more of 2-ethanol; and 25 mg or more of phosphatidylcholine.

### Example 2: Vape Formulations

KLM9500 is a 1:1 ratio of magnolol and h.

### Formulation A (standard vape solution)

First, 1 kg of KLM9500 is used to form a basic solution:

Dissolve 1 kg of KLM9500 in 1,333 L of propylene glycol and 1,333 L of glycerine (vegetable glycerin). This produces 177,777 bottles (15 ml each) at a concentration of 0.375 mg/ml of KLM9500 per dose (1.6 ml dose), which is equivalent to 2 capsules for administration.

### Formulation B (standard vape solution)

Step 1: 5 g of K1M-9500 dissolved at room temperature into 100 ml of pharmaceutical grade propylene glycol.
Step 2: 15 ml of above solution is then dissolved in a 985 ml of propylene glycol and 1 L of glycerine in preferred ratio (50/50 used in sample). This produces a concentration of KLM-9500 of 0.6 mg/1.6 ml of KLM-9500 (in some embodiments, this is equivalent to two capsules)

### Formulation C (tincture vape solution)

1 kg KLM9500 is dissolved in 667 L of propylene glycol. This produces 44,444 bottles (15 ml each) having a concentration of 1.5 mg/ml. One bottle of tincture (15 ml) at 1.5 mg KLM9500 per ml propylene glycol will produce 4 bottles of standard mix at 0.6 mg KLM9500 per 1.6 ml dose.

### Formulation D (maximum solubility - tincture x4)

1.5 mg/1 ml of KLM-9500 (this would be equivalent to 2.5 capsules per ml)

0.6 mg of KLM9500 is a particularly useful dose. It some cases, it is administered once or twice per day, In some cases, a 1.2 mg dose is administered daily (or two 1.2 mg doses administered concurrently).

A non-parametric Friedman within subjects ANOVA was conducted to compare the change in the CAR across the study period for the treatment group and control group. Statistical analysis indicated there was no significant change in the CAR over the study period for the control group, χ2 (3, N=14)=1.63, p=0.653. However, in the treatment group, statistical analyses control group, χ2 (3, N=14)=1.63, p=0.653. However, in the treatment group, statistical analyses indicated a trend towards significance, χ2 (3, N= 16)=4.21, p=0.240. Further examination of the CAR showed that it tended to decrease following Baseline week for the treatment group, with this drop in CA.R maintained throughout the study. However, the control group showed the opposite pattern, with the CAR increasing following the Baseline week and throughout the study period.

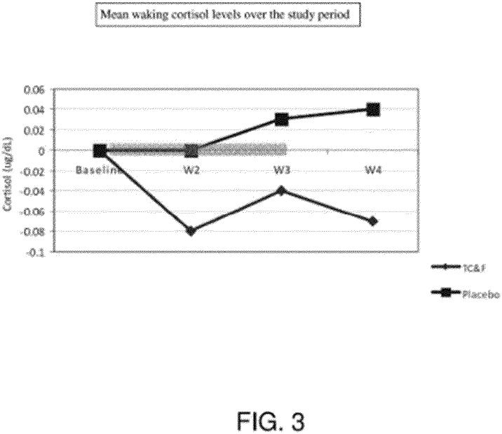

Closer examination of cortisol levels from saliva samples provided immediately upon waking was performed, as the process of awakening results in HPA-axis activity, often characterized by a marked increase in cortisol levels, therefore providing an accurate measure of the stress response. Mean waking cortisol levels over the study period for the treatment group and control group are presented graphically in FIG. 3.

A non-parametric Friedman one-way within subjects ANOVA found that there was no significant difference in waking cortisol levels across the study period for the control group χ2 (3, N=14)=0.86, p=0.836. However, there was a significant effect for the study sampling period for waking cortisol levels for the treatment group χ2 (3, N=17)=8.08, p=0.044. Pairwise comparisons with non-parametric t-tests (Wilcoxon Signed Rank Test) indicated that the amount of waking cortisol detected in the baseline condition was significantly greater than the amount detected in experimental week 1 (Z=- 2.33), p=0.02) and follow-up week 4 (Z=-2.20, p=0.028) of the treatment group.

### Salivary Alpha-Amylase

A non-parametric Friedman one-way within subjects ANOVA was conducted to compare the levels of alpha-amylase detected from evening saliva samples over the study period for the treatment and control groups. For both groups, there were no significant differences in salivary alpha-amylase levels over the study period, χ2 (3, N= 18)=6.02, p=0.111 and χ2 (3, N=15)=4.36, p=0.225, respectively.

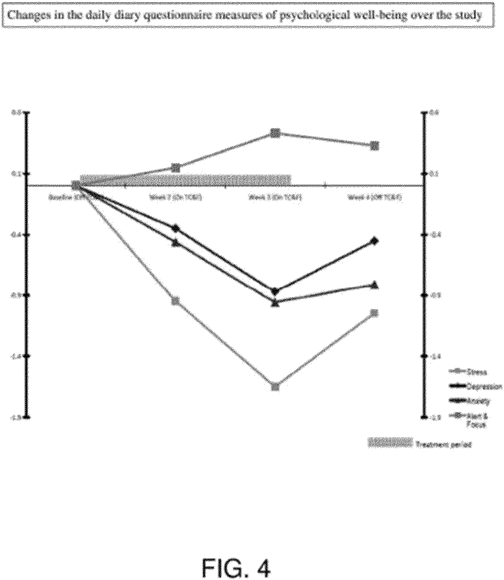

A non-parametric Friedman within subjects ANOVA was conducted to compare the changes in the daily diary questionnaire measures of psychological well-being over the study. For the control group, statistical analysis found there was no significant change in the daily measures of stress, depression, anxiety, and alert and focus over the study period, (χ2 (3, N=16)=7.53, p=0.057, χ2 (3, N=16)=6.21, p=0.102, χ2 (3, N=16)=0.95, p=0.814, χ2 (3, N=16)=3.38, p=0.337, respectively. However, in the treatment group, statistical analyses found a significant difference in the daily measure of stress (x2 (3, N=18)=11.01, p=0.012). There was no significant difference in the daily measures of depression, anxiety and alert-and-focus in the treatment group (χ2 (3, N=18)=5.20, p=0.157, χ2 (3, N=18)=4.24, p=0.237. χ2 (3, N=18)=0.83, p=0.843, respectively). See FIG. 4. Examination of these daily measures showed an interesting pattern for the ill being measures of stress, depression, and anxiety, with levels continuing to decrease up until experimental week 3, followed by a rise in levels in the follow-up week of the treatment group. The well-being measure of alert-and-focus remained relatively stable over the study period.

What is claimed is:

1. A composition for a reduction of a level of cortisol in a subject, the composition comprising:
an olive oil comprising hydroxytyrosol;
a phospholipid;
an amount of cannabidiol effective to modify the subject’s metabolism;
an amount of one or both of magnolol and honokiol effective to provide an antiangiogenic, anti-inflammatory effect in a patient receiving the composition;
a carotenoid;
an Ashwagandha extract; and
a withanolide compound.

2. The composition of claim 1, wherein the phospholipid comprises lecithin.

3. The composition of claim 1, wherein the phospholipid comprises phosphatidylserine.

4. The composition of claim 1, further comprising at least one additional compound selected from the group consisting of a proanthocyanidin, CMT oil, an omega-3- fatty acid, ethanol, and an antioxidant.

5. The composition of claim 1, further comprising at least one mineral complex selected from the group consisting of calcium, copper, iron, iodine, lithium, magnesium, manganese, potassium, vanadium, and zinc.

6. The composition of claim 4, wherein a mineral complex comprises at least one of magnesium, calcium, zinc, and iron.

7. The composition of claim 5, wherein the at least one of magnesium, calcium, zinc, and iron act as Krebs Cycle intermediates.

8. The composition of claim 1, further comprising at least one B-complex vitamin selected from the group consisting of Vitamin B1, Vitamin B2, Vitamin B3, Vitamin B5 and Vitamin B6.

9. The composition of claim 1, further comprising an herb Luo Han Guo (Siraitia grosvenorii).

10. The composition of claim 1, comprising 20 to 30 wt% of the Ashwagandha extract having withanolides at more than 5 wt%; 45 to 55 wt% of lecithin; 5 to 15 wt% phosphatidylserine; 5 to 15% of the olive oil containing the hydroxytyrosol at 400-500 umol hydroxytyrosol/kg; and 1 to 3 wt% magnolia bark or magnolia bark extract, excluding solvent or carrier liquid.

11. The composition of claim 9, comprising 125 mg of the Ashwagandha extract, 250 mg of lecithin, 50 mg of phosphatidylserine, 50 mg of the olive oil supplemented with 400 to 500 micromol of hydroxytyrosol/kg of the olive oil, and 7 mg of magnolia bark extract.

12. A method of reducing a cortisol level in a subject, comprising administering the composition of claim 1 to the subject in a therapeutically effective amount, thereby lowering the cortisol level in the subject.

13. The method of claim 12, wherein the cortisol level that is lowered is a waking cortisol level.

14. The method of claim 12, further comprising attenuating a Cortisol Awakening Response (CAR) in the subject.

15. A method of treating an ailment in a subject in need thereof, comprising administering to the subject the composition of claim 1 in a therapeutically effective amount to treat the ailment in the subject.

16. The method of claim 15, wherein the ailment comprises at least one of stress, anxiety, depression, and insomnia.

17. A method of decreasing body weight of a subject, comprising administering to the subject the composition of claim 1 in a therapeutically effective amount to reduce the body weight of the subject.

18. A method of increasing alertness, focus, or both in a subject, comprising administering to the subject the composition of claim 1 in a therapeutically effective amount to increase alertness, focus, or both, in the subject.
